(19) United States
(12) Patent Application Publication
(10) Pub. No.: US 2021/0151124 A1
(43) Pub. Date: May 20, 2021

(54) GENETIC VARIATION IDENTIFICATION METHOD, GENETIC VARIATION IDENTIFICATION APPARATUSES, AND STORAGE MEDIUM

(71) Applicant: BEIJING SENSETIME TECHNOLOGY DEVELOPMENT CO., LTD., Beijing (CN)

(72) Inventor: Zhiqiang HU, Beijing (CN)

(73) Assignee: BEIJING SENSETIME TECHNOLOGY DEVELOPMENT CO., LTD., Beijing (CN)

(21) Appl. No.: 17/162,465

(22) Filed: Jan. 29, 2021

Related U.S. Application Data
(63) Continuation of application No. PCT/CN2019/089504, filed on May 31, 2019.

(30) Foreign Application Priority Data
Mar. 29, 2019 (CN) 201910252747.9

(51) Int. Cl. G16B 20/20; G16B 30/10

(52) U.S. Cl. CPC G16B 20/20; G16B 30/10

(57) ABSTRACT

The present disclosure relates to a genetic variation identification method, genetic variation identification apparatuses, and a storage medium. The method includes: obtaining at least one gene sequencing read corresponding to a genetic variation candidate site; obtaining base arrangement features of the genetic variation candidate site; determining non-base arrangement features of the genetic variation candidate site based on non-base arrangement information of the at least one gene sequencing read within a preset site interval, where the non-base arrangement features remain unchanged after a base arrangement order changes; and identifying genetic variation of the genetic variation candidate site based on the base arrangement features and the non-base arrangement features of the genetic variation candidate site. In embodiments of the present disclosure, in consideration of the feature that non-base arrangement features are not constrained by a base arrangement order, pseudo-genetic variation caused by germ-line genetic variation and interferences such as noise and errors can be better screened out, so that genetic variation is better identified and accuracy of genetic variation identification is improved.

### CROSS-REFERENCE TO RELATED APPLICATIONS

The present disclosure is a bypass continuation of and claims priority under 35 U.S.C. § 111(a) to PCT Application. No. PCT/CN2019/089504, filed on May 31, 2019, which claims priority to Chinese Patent Application No. 201910252747.9, filed with the Chinese Patent Office on Mar. 29, 2019 and entitled “GENETIC VARIATION IDENTIFICATION METHOD, GENETIC VARIATION IDENTIFICATION APPARATUSES, AND STORAGE MEDIUM”, which are incorporated herein by reference in their entireties.

### TECHNICAL FIELD

The present disclosure relates to the technical field of computers, and in particular, to a genetic variation identification method, genetic variation identification apparatuses, and a storage medium.

### BACKGROUND

With the development of biotechnology, human gene sequencing can be achieved by the gene sequencing technology, and analysis of base sequences can be taken as the basis for further genetic research and transformation. At present, compared with the first-generation testing technology, the second-generation gene sequencing technology has greatly improved the efficiency of gene sequencing, reduced the costs of gene sequencing, and maintained the accuracy of gene sequencing. The first-generation testing technology may take 3 years to complete sequencing of a human genome, while the second-generation sequencing technology can shorten the time to 1 week.

### SUMMARY

In this regard, the present disclosure provides a genetic variation identification technical solution.

A genetic variation identification method is provided according to one aspect of the present disclosure, including:

obtaining at least one gene sequencing read corresponding to a genetic variation candidate site; obtaining base arrangement features of the genetic variation candidate site; determining non-base arrangement features of the genetic variation candidate site based on non-base arrangement information of the at least one gene sequencing read within a preset site interval, where the non-base arrangement features remain unchanged after a base arrangement order changes; and identifying genetic variation of the genetic variation candidate site based on the base arrangement features and the non-base arrangement features of the genetic variation candidate site.

In one possible implementation, obtaining the base arrangement features of the genetic variation candidate site includes:

determining the preset site interval where the genetic variation candidate site is located; and obtaining the base arrangement features of the genetic variation candidate site according to base arrangement information of a reference genome within the preset site interval, where the base arrangement features are used for representing the base arrangement order.

In one possible implementation, determining the non-base arrangement features of the genetic variation candidate site based on the non-base arrangement information of the at least one gene sequencing read within the preset site interval includes:

obtaining the non-base arrangement information of the at least one gene sequencing read at each site within the preset site interval; and determining the non-base arrangement features of the genetic variation candidate site based on the non-base arrangement information at each site within the preset site interval.

In one possible implementation, determining the non-base arrangement features of the genetic variation candidate site based on the non-base arrangement information at each site within the preset site interval includes:

determining, from the gene sequencing reads, a first gene sequencing read having a consistent base type with the reference genome at the genetic variation candidate site; and determining the non-base arrangement features of the genetic variation candidate site according to the number of first gene sequencing reads corresponding to each site within the preset site interval.

In one possible implementation, determining the non-base arrangement features of the genetic variation candidate site based on the non-base arrangement information at each site within the preset site interval includes:

determining, from the gene sequencing reads, a first gene sequencing read having a consistent base type with the reference genome at the genetic variation candidate site; determining the number of first gene sequencing reads having inconsistent base types with the reference genome at each site within the preset site interval, as the number of first gene sequencing reads to which variation occurs; and determining the non-base arrangement features of the genetic variation candidate site according to the number of first gene sequencing reads to which variation occurs.

In one possible implementation, determining the non-base arrangement features of the genetic variation candidate site based on the non-base arrangement information at each site within the preset site interval includes:

determining, from the gene sequencing reads, a second gene sequencing read having a base type at the genetic variation candidate site consistent with a variant base type of the genetic variation candidate site; and determining the non-base arrangement features of the genetic variation candidate site according to the number of second gene sequencing reads corresponding to each site within the preset site interval.

In one possible implementation, determining the non-base arrangement features of the genetic variation candidate site based on the non-base arrangement information at each site within the preset site interval includes:

determining, from the gene sequencing reads, a second gene sequencing read having a base type at the genetic variation candidate site consistent with a variant base type of the genetic variation candidate site; determining the number of second gene sequencing reads having inconsistent base types with the reference genome at each site within the preset site interval, as the number of second gene sequencing reads to which variation occurs; and determining the non-base arrangement features of the genetic variation candidate site according to the number of second gene sequencing reads to which variation occurs.

In one possible implementation, determining the non-base arrangement features of the genetic variation candidate site based on the non-base arrangement information at each site within the preset site interval includes:

determining a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; and determining the non-base arrangement features of the genetic variation candidate site according to the number of third gene sequencing reads corresponding to each site within the preset site interval.

In one possible implementation, determining the non-base arrangement features of the genetic variation candidate site based on the non-base arrangement information at each site within the preset site interval includes:

determining a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; determining the number of third gene sequencing reads having inconsistent base types with the reference genome at each site within the preset site interval, as the number of third gene sequencing reads to which variation occurs; and determining the non-base arrangement features of the genetic variation candidate site according to the number of third gene sequencing reads to which variation occurs.

In one possible implementation, determining the non-base arrangement features of the genetic variation candidate site based on the non-base arrangement information at each site within the preset site interval includes:

determining, from the at least one gene sequencing read, a gene sequencing read derived from a normal cell; and determining the non-base arrangement features of the genetic variation candidate site based on non-base arrangement information of the gene sequencing read of the normal cell at each site within the preset site interval.

In one possible implementation, determining the non-base arrangement features of the genetic variation candidate site based on the non-base arrangement information at each site within the preset site interval includes:

determining, from the at least one gene sequencing read, a gene sequencing read derived from a diseased cell; and determining the non-base arrangement features of the genetic variation candidate site based on non-base arrangement information of the gene sequencing read of the diseased cell at each site within the preset site interval.

In one possible implementation, identifying the genetic variation of the genetic variation candidate site based on the base arrangement features and the non-base arrangement features of the genetic variation candidate site includes:

obtaining a feature matrix of the genetic variation candidate site according to the base arrangement features and the non-base arrangement features of the genetic variation candidate site, where first dimension features of the feature matrix correspond to the base arrangement features and the non-base arrangement features of the genetic variation candidate site, and second dimension features of the feature matrix correspond to sites within the preset site interval; and identifying the genetic variation of the genetic variation candidate site according to the feature matrix of the genetic variation candidate site.

In one possible implementation, identifying the genetic variation of the genetic variation candidate site according to the feature matrix of the genetic variation candidate site includes:

obtaining a variation value for occurrence of genetic variation at the genetic variation candidate site according to the feature matrix of the genetic variation candidate site; and

in the case that the variation value is greater than or equal to a preset threshold, determining that genetic variation exists at the genetic variation candidate site.

In one possible implementation, obtaining the feature matrix of the genetic variation candidate site according to the base arrangement features and the non-base arrangement features of the genetic variation candidate site includes:

generating a feature vector of each first dimension feature of the preset site interval according to the base arrangement features and the non-base arrangement features of the genetic variation candidate site; determining base arrangement feature vectors formed by the base arrangement features in the feature vectors; and randomly ranking the base arrangement feature vectors to obtain the feature matrix of the genetic variation candidate site.

In one possible implementation, obtaining at least one gene sequencing read corresponding to the genetic variation candidate site includes:

obtaining gene sequencing reads obtained by performing gene sequencing on a somatic gene; comparing base sequences of the gene sequencing reads with a base sequence of the reference genome to obtain a comparison result; determining the genetic variation candidate site, at which a gene abnormality exists, of the somatic gene according to the comparison result; and obtaining at least one gene sequencing read corresponding to the genetic variation candidate site.

A genetic variation identification apparatus is provided according to another aspect of the present disclosure, including:

a first obtaining module, configured to obtain at least one gene sequencing read corresponding to a genetic variation candidate site; a second obtaining module, configured to obtain base arrangement features of the genetic variation candidate site; a determining module, configured to determine non-base arrangement features of the genetic variation candidate site based on non-base arrangement information of the at least one gene sequencing read within a preset site interval, where the non-base arrangement features remain unchanged after a base arrangement order changes; and an identifying module, configured to identify genetic variation of the genetic variation candidate site based on the base arrangement features and the non-base arrangement features of the genetic variation candidate site.

In one possible implementation, the second obtaining module includes:

a first determining sub-module, configured to determine the preset site interval where the genetic variation candidate site is located; and

a second determining sub-module, configured to obtain the base arrangement features of the genetic variation candidate site according to base arrangement information of a reference genome within the preset site interval, where the base arrangement features are used for representing the base arrangement order.

In one possible implementation, the determining module includes:

a first obtaining sub-module, configured to obtain non-base arrangement information of the at least one gene sequencing read at each site within the preset site interval; and a third determining sub-module, configured to determine the non-base arrangement features of the genetic variation candidate site based on the non-base arrangement information at each site within the preset site interval.

In one possible implementation, the third determining sub-module is specifically configured to:

determine, from the gene sequencing reads, a first gene sequencing read having a consistent base type with the reference genome at the genetic variation candidate site; and determine the non-base arrangement features of the genetic variation candidate site according to the number of first gene sequencing reads corresponding to each site within the preset site interval.

In one possible implementation, the third determining sub-module is specifically configured to:

determine, from the gene sequencing reads, a first gene sequencing read having a consistent base type with the reference genome at the genetic variation candidate site; determine the number of first gene sequencing reads having inconsistent base types with the reference genome at each site within the preset site interval, as the number of first gene sequencing reads to which variation occurs; and determine the non-base arrangement features of the genetic variation candidate site according to the number of first gene sequencing reads to which variation occurs.

In one possible implementation, the third determining sub-module is specifically configured to:

determine, from the gene sequencing reads, a second gene sequencing read having a base type at the genetic variation candidate site consistent with a variant base type of the genetic variation candidate site; and determine the non-base arrangement features of the genetic variation candidate site according to the number of second gene sequencing reads corresponding to each site within the preset site interval.

In one possible implementation, the third determining sub-module is specifically configured to:

determine, from the gene sequencing reads, a second gene sequencing read having a base type at the genetic variation candidate site consistent with a variant base type of the genetic variation candidate site; determine the number of second gene sequencing reads having inconsistent base types with the reference genome at each site within the preset site interval, as the number of second gene sequencing reads to which variation occurs; and determine the non-base arrangement features of the genetic variation candidate site according to the number of second gene sequencing reads to which variation occurs.

In one possible implementation, the third determining sub-module is specifically configured to:

determine a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; and determine the non-base arrangement features of the genetic variation candidate site according to the number of third gene sequencing reads corresponding to each site within the preset site interval.

In one possible implementation, the third determining sub-module is specifically configured to:

determine a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; determine the number of third gene sequencing reads having inconsistent base types with the reference genome at each site within the preset site interval, as the number of third gene sequencing reads to which variation occurs; and determine the non-base arrangement features of the genetic variation candidate site according to the number of third gene sequencing reads to which variation occurs.

In one possible implementation, the third determining sub-module is specifically configured to:

determine, from the at least one gene sequencing read, a gene sequencing read derived from a normal cell; and determine the non-base arrangement features of the genetic variation candidate site based on non-base arrangement information of the gene sequencing read of the normal cell at each site within the preset site interval.

In one possible implementation, the third determining sub-module is specifically configured to:

determine, from the at least one gene sequencing read, a gene sequencing read derived from a diseased cell; and determine the non-base arrangement features of the genetic variation candidate site based on non-base arrangement information of the gene sequencing read of the diseased cell at each site within the preset site interval.

In one possible implementation, the identifying module includes:

a generating sub-module, configured to obtain a feature matrix of the genetic variation candidate site according to the base arrangement features and the non-base arrangement features of the genetic variation candidate site, where first dimension features of the feature matrix correspond to the base arrangement features and the non-base arrangement features of the genetic variation candidate site, and second dimension features of the feature matrix correspond to sites within the preset site interval; and an identifying sub-module, configured to identify genetic variation of the genetic variation candidate site according to the feature matrix of the genetic variation candidate site.

In one possible implementation, the identifying sub-module is specifically configured to:

obtain a variation value for occurrence of genetic variation at the genetic variation candidate site according to the feature matrix of the genetic variation candidate site; and

in the case that the variation value is greater than or equal to a preset threshold, determine that genetic variation exists at the genetic variation candidate site.

In one possible implementation, the generating sub-module is specifically configured to:

generate a feature vector of each first dimension feature of the preset site interval according to the base arrangement features and the non-base arrangement features of the genetic variation candidate site; determine base arrangement feature vectors formed by the base arrangement features in the feature vectors; and randomly rank the base arrangement feature vectors to obtain the feature matrix of the genetic variation candidate site.

In one possible implementation, the first obtaining module includes:

a second obtaining sub-module, configured to obtain gene sequencing reads obtained by performing gene sequencing on a somatic gene; a comparing sub-module, configured to compare base sequences of the gene sequencing reads with a base sequence of the reference genome to obtain a comparison result; a fourth determining sub-module, configured to determine the genetic variation candidate site, at which a gene abnormality exists, of the somatic gene according to the comparison result; and a third obtaining sub-module, configured to obtain at least one gene sequencing read corresponding to the genetic variation candidate site.

A genetic variation identification apparatus is provided according to another aspect of the present disclosure, including: a processor; and a memory configured to store processor-executable instructions, where the processor is configured to execute the forgoing method.

A non-volatile computer-readable storage medium is provided according to another aspect of the present disclosure, having computer program instructions stored thereon, where when the computer program instructions are executed by a processor, the foregoing method is implemented.

According to the genetic variation identification solution provided in embodiments of the present disclosure, at least one gene sequencing read corresponding to a genetic variation candidate site is obtained; base arrangement features of the genetic variation candidate site are obtained; non-base arrangement features of the genetic variation candidate site are determined based on base arrangement information of the at least one gene sequencing read within a preset site interval; and thus genetic variation of the genetic variation candidate site is identified based on the base arrangement features and the non-base arrangement features of the genetic variation candidate site. Here, the non-base arrangement features remain unchanged after a base arrangement order changes, i.e., it is recognized that the non-base arrangement feature has a base arrangement invariance property. Therefore, during identification of genetic variation of the genetic variation candidate site, the feature that genetic variation of the genetic variation candidate site is not constrained by the base arrangement order is taken into consideration, so that pseudo-genetic variation caused by germ-line genetic variation and interferences such as noise and errors can be better screened out, so that genetic variation is better identified and accuracy of genetic variation identification is improved.

It should be understood that the above general description and the following detailed description are merely exemplary and explanatory and are not intended to limit the present disclosure.

The other features and aspects of the present disclosure can be described more clearly according to the detailed descriptions of the exemplary embodiments in the accompanying drawings.

### BRIEF DESCRIPTION OF THE DRAWINGS

The accompanying drawings included in the description and constituting a part of the description illustrate the exemplary embodiments, features, and aspects of the present disclosure together with the description, and are used for explaining the principles of the present disclosure.

FIG. 1 is a flowchart of a genetic variation identification method according to one embodiment of the present disclosure.

FIG. 2 is a flowchart of obtaining at least one gene sequencing read corresponding to a genetic variation candidate site according to one embodiment of the present disclosure.

FIG. 3 is a flowchart of a process of obtaining base arrangement features of a genetic variation candidate site according to one embodiment of the present disclosure.

FIG. 4 is a flowchart of a process of determining non-base arrangement features of a genetic variation candidate site according to one embodiment of the present disclosure.

FIG. 5 is a flowchart of a process of identifying genetic variation of a genetic variation candidate site according to one embodiment of the present disclosure.

FIG. 6 is a flowchart of a process of obtaining a feature matrix of a genetic variation candidate site according to one embodiment of the present disclosure.

FIG. 7 is a block diagram of a genetic variation identification apparatus according to embodiments of the present disclosure.

FIG. 8 is a block diagram of a genetic variation identification apparatus 1900 according to one exemplary embodiment.

### DETAILED DESCRIPTION

The various exemplary embodiments, features, and aspects of the present disclosure are described below in detail with reference to the accompanying drawings. The same reference numerals in the accompanying drawings represent elements having the same or similar functions. Although the various aspects of the embodiments are illustrated in the accompanying drawings, unless stated particularly, it is not required to draw the accompanying drawings in proportion.

The special word “exemplary” here means “used as examples, embodiments, or descriptions”. Any “exemplary” embodiment given here is not necessarily construed as being superior to or better than other embodiments.

The term “and/or” in the text only describes an association relationship between associated objects, and indicates that there are three kinds of relationships, for example, A and/or B indicates three situations, i.e., A exists alone, A and B exist simultaneously, and B exists alone. In addition, the term “at least one” in the text indicates any one of multiple objects or any combination of at least two of multiple objects, for example, including at least one of A, B, or C indicates including any one or more elements selected from a set formed by A, B, and C.

In addition, numerous details are given in specific implementations below for the purpose of better explaining the present disclosure. It should be understood by persons skilled in the art that the present disclosure can still be implemented even without some of those details. In some examples, methods, means, elements, and circuits that are well known to persons skilled in the art are not described in detail so that the principle of the present disclosure becomes apparent.

According to the genetic variation identification solution provided in embodiments of the present disclosure, at least one genetic sequencing read corresponding to a genetic variation candidate site is obtained, and thus genetic variation of the genetic variation candidate side is identified by using the at least one gene sequencing read. In the genetic variation identification process, base arrangement features of the genetic variation candidate site are determined, and non-base arrangement features of the genetic variation candidate site are determined according to base arrangement information of the at least one gene sequencing read within a preset site interval; and then the genetic variation of the genetic variation candidate site is identified through the base arrangement features and the non-base arrangement features. The non-base arrangement features here remain unchanged after a base arrangement order changes, i.e., it is recognized that whether the genetic variation of the genetic variation candidate site is true variation is not affected by the base arrangement order. Therefore, during identification of the genetic variation of the genetic variation candidate site, in consideration of base arrangement invariance of genetic data, accuracy of genetic variation identification is improved.

In the related art, genetic variation identification is generally performed by using conventional machine learning methods such as a support vector machine and random forest. Although those methods are simple to implement, the effect of genetic variation identification will be bottlenecked as the amount of genetic data has increased to a certain extent. There are also some related technologies genetic variation is identified by a neural network by a deep learning method. However, features extracted by the neural network are usually related to the base arrangement order, and thus a different identification result is obtained as long as the base arrangement order differs slightly, thereby causing a neural network over-fitting problem. However, according to the genetic variation identification solution provided in embodiments of the present disclosure, in consideration of the base arrangement invariance of genetic data, non-base arrangement features of the genetic variation candidate site are extracted by using a genetic variation identification model, so that the obtained identification result will not be affected by the base arrangement order, thereby improving robustness of the genetic variation identification model, mitigating the over-fitting problem, and reducing the difficulty in genetic variation identification model training. The following embodiments will explain the genetic variation identification process in detail.

FIG. 1 is a flowchart of a genetic variation identification method according to one embodiment of present disclosure. The genetic variation identification method is executed by a genetic variation identification apparatus or other processing devices, where the genetic variation identification apparatus is a User Equipment (UE), a mobile device, a user terminal, a terminal, a cellular phone, a cordless phone, or a Personal Digital Assistant (PDA), a handheld device, a computing device, an in-vehicle device, a wearable device, or the like. Alternatively, the genetic variation identification apparatus is a server. In some possible implementations, the genetic variation identification method may be implemented by a processor invoking computer-readable instructions stored in a memory. As shown in FIG. 1, the genetic variation identification method includes the following steps.

At step 11, at least one gene sequencing read corresponding to a genetic variation candidate site is obtained.

In embodiments of the present disclosure, the genetic variation identification apparatus obtains gene sequencing reads obtained from gene sequencing, and then obtains, from the gene sequencing reads obtained from gene sequencing, the at least one gene sequencing read corresponding to the genetic variation candidate site. The gene sequencing read here may be understood as a base sequence labeled with a base type after gene sequencing, and the lengths of the gene sequencing reads may be identical or different. In the case that the lengths are different, the length of each gene sequencing read may fall within a preset length range, so as to ensure that the lengths of the gene sequencing reads are close to each other. The base type includes cytosine (C), guanine (G), adenine (A), and thymine (T), and thus the gene sequencing read may include an AGCT base sequence. The genetic variation candidate site here may be an abnormal site of a base sequence. The site of a base sequence may represent the position of the base sequence, and for each site, there may be at least one gene sequencing read, i.e., at least one gene sequencing read obtained from gene sequencing may exist at the same site. Accordingly, the genetic variation candidate site corresponds to at least one gene sequencing read, where the at least one gene sequencing read all covers the site. There may be at least one gene variation candidate site, and each gene variation candidate site may correspond to at least one gene sequencing read. In order to facilitate understanding, the explanations in the embodiments of the present disclosure are made for one genetic variation candidate site.

At step 12, base arrangement features of the genetic variation candidate site are obtained.

In the embodiments of the present disclosure, the base arrangement features of the genetic variation candidate site are extracted according to gene arrangement information of the genetic variation candidate site by using a gene variation identification model. The base arrangement information here is the information related to a base arrangement order, for example, if the base sequences of a certain gene sequencing rad within a certain site interval are A, C, G, and T in sequence, the base arrangement information is ACGT. The base arrangement information includes information within a preset site interval, such as a base type of a reference genome, the number of genes of each base type, the number of missing genes of each base type, and the number of inserted genes of each base type. The base arrangement features obtained from the base arrangement information are related to the base arrangement order.

At step 13, non-base arrangement features of the genetic variation candidate site are determined based on non-base arrangement information of the at least one gene sequencing read within a preset site interval, where the non-base arrangement features remain unchanged after a base arrangement order changes.

In the embodiments of the present disclosure, after obtaining the at least one gene sequencing read corresponding to the genetic variation candidate site, the non-base arrangement information of the at least one gene sequencing read corresponding to the genetic variation candidate site is extracted within the preset site interval, and the non-base arrangement features of the genetic variation candidate site are generated according to the extracted non-base arrangement information. The non-base arrangement information is the information not constrained by the base arrangement order. Therefore, the non-base arrangement features of the genetic variation candidate site are determined according to the non-base arrangement information of the at least one gene sequencing read within the preset site interval. Here, the non-base arrangement information includes information having base arrangement invariance, such as the number of gene sequencing reads corresponding to a site and the number of gene sequencing reads to which variation occurs at the site.

Here, during extraction of the non-base arrangement information, several gene sequencing reads corresponding to the genetic variation candidate site are selected randomly, and the non-base arrangement information of the several gene sequencing reads selected randomly is extracted. Alternately, the non-base arrangement information of each gene sequencing read corresponding to the genetic variation candidate site is extracted. When extracting the non-base arrangement information of the at least one gene sequencing read within the present site interval, non-base arrangement information of the at least one gene sequencing read at each site within the preset site interval is extracted. Alternately, several adjacent sites within the preset site interval are selected randomly, and non-base arrangement information of the at least one gene sequencing read at the several adjacent sites is extracted. When determining the non-base arrangement features of the genetic variation candidate site, a genetic variation identification model obtained based on neural network training is used.

At step 14, genetic variation of the genetic variation candidate site is identified based on the base arrangement features and the non-base arrangement features of the genetic variation candidate site.

In implementations of the present disclosure, after determining the base arrangement features and the non-base arrangement features, a feature matrix of the genetic variation candidate site is obtained from the base arrangement features and the non-base arrangement features, and generic variation of the genetic variation candidate site is identified by using the feature matrix. For example, the foregoing genetic variation identification model is used for determining whether gene variation of the genetic variation candidate site is true variation caused by a disease or pseudo variation of a base sequence abnormality caused by noise or the like. Here, the obtained feature matrix of the genetic variation candidate site is a two-dimensional feature matrix, the size of the feature matrix is the product of the number of feature vectors and the size of the preset site interval, and the feature vector is generated based on the base arrangement feature and the non-base arrangement feature. Whether genetic variation of a variation candidate site is true genetic variation caused is not affected by the base arrangement order, but more affected by a gene environment where the genetic variation candidate site is located. For example, it is affected by gene environments such as a variant gene exists at another side near the genetic variation candidate site. Therefore, the arrangement order of the feature vectors corresponding to the base arrangement features in the obtained feature matrix is not limited, and the arrangement order of the feature vectors of the base arrangement features in the feature matrix can change randomly, thereby improving efficiency and accuracy of genetic variation identification.

In the embodiments of the present disclosure, genetic variation of a genetic variation candidate site is identified according to base arrangement features and non-base arrangement features of the genetic variation candidate site, so that genetic variation can be better identified in consideration of the base arrangement invariance of genetic variation. When identifying genetic variation of the genetic variation candidate site, at least one gene sequencing read corresponding to the genetic variation candidate site is obtained. The embodiments of the present disclosure also provide a process of obtaining at least one gene sequencing read corresponding to a genetic variation candidate site.

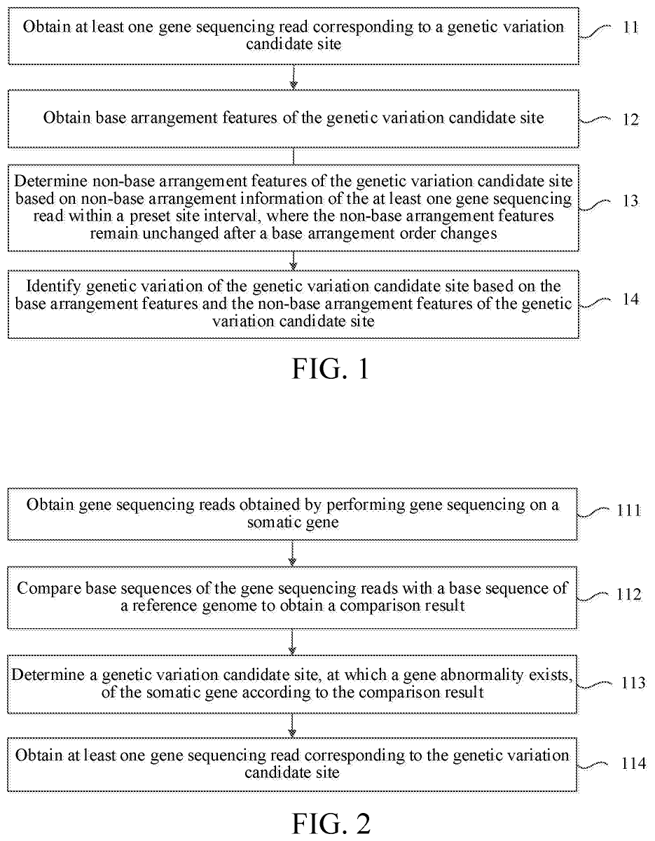

FIG. 2 is a flowchart of obtaining the at least one gene sequencing read corresponding to the genetic variation candidate site according to one embodiment of the present disclosure. In one possible implementation, obtaining the at least one gene sequencing read corresponding to the genetic variation candidate site may include the following steps.

At step 111, Gene sequencing reads obtained by performing gene sequencing on a somatic gene are obtained.

Here, at least one gene sequencing read may be obtained by performing gene sequencing on the somatic gene, and the gene sequencing read may be a sequence for labeling a base type of the somatic gene. After performing gene sequencing on the somatic gene, the base type of each gene in the gene sequencing read may be obtained, and gene position information of a site where each gene in the gene sequencing read is located. The same site may correspond to at least one gene sequencing read.

In one possible implementation, at least one gene sequencing read may be obtained by performing gene sequencing on the somatic gene, and the gene sequencing read obtained from gene sequencing may be pretreated, where a method for the pretreatment may include cross-contamination screening, sequencing quality screening, comparison quality screening, read length abnormality screening, etc. By means of the pretreatment, a gene sequencing read in cross-contamination may be screened out, and a gene sequencing read having low sequencing quality, low comparison quality, or an abnormal read length may be screened out.

At step 112, base sequences of the gene sequencing reads are compared with a base sequence of the reference genome to obtain a comparison result.

In embodiments of the present disclosure, after obtaining the gene sequencing reads obtained by performing gene sequencing on the somatic gene, the base sequences of the obtained gene sequencing reads may be compared with the base sequence of the reference gene at the same site to obtain the comparison result. For example, each gene sequencing read obtained by performing gene sequencing is compared with the reference genome at the same site in terms of base sequence, and a site at which the base sequences of the gene sequencing reads are different from the base sequence of the reference genome is determined. Alternately, at least one gene sequencing read of the same site is compared with the reference genome at the same site in terms of base sequence, and a site at which the base sequence of the at least one gene sequencing read is different from the base sequence of the reference genome is determined. Here, the reference genome may be the base sequence labeled with a correct base sequence.

At step 113, the genetic variation candidate site, at which a gene abnormality exists, of the somatic gene is determined according to the comparison result.

In the embodiments of the present disclosure, a site at which the gene sequencing read is different from the reference genome in terms of base sequence is determined according to the comparison result. If the ratio of the gene sequencing reads, to which variation occurs at the site, in the at least one gene sequencing read corresponding to the site is greater than a preset ratio, the site is determined as the genetic variation candidate site, otherwise, it is recognized that the site is not the genetic variation candidate site. The difference between the gene sequencing read and the reference genome in terms of base sequence at the site may be caused by a sequencing error. In this case, a base sequence abnormality phenomenon caused by the gene sequencing error may be reduced.

At step 114, at least one gene sequencing read corresponding to the genetic variation candidate site is obtained.

In the embodiments of the present disclosure, after determining the genetic variation candidate site, at least one gene sequencing read corresponding to the genetic variation candidate site may be obtained. At genetic variation candidate site, the base sequence of the at least one gene sequencing read corresponding to each genetic variation candidate site may be different from the base sequence of the reference genome at the same site. Here, at least one genetic variation candidate site may be included.

Through the foregoing process of obtaining at least one gene sequencing read corresponding to the genetic variation candidate site, not only can the gene variation candidate site be determined more accurately, but also at least one gene sequencing read corresponding to the genetic variation candidate site can be determined from the gene sequencing reads obtained from gene sequencing.

In the embodiments of the present disclosure, base arrangement features of a genetic variation candidate site may be determined according to base arrangement information of at least one gene sequencing read corresponding to the genetic variation candidate site, thereby enabling data enhancement processing for gene identification according to the base arrangement features when identifying genetic variation of the genetic variation candidate site. A process of determining the base arrangement features of the genetic variation candidate site is explained in detail in an example below.

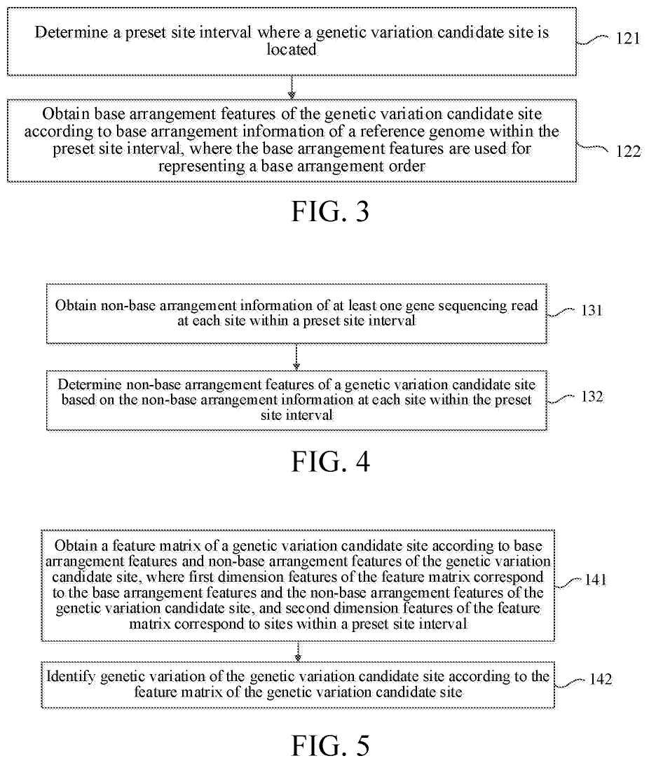

FIG. 3 is a flowchart of a process of obtaining the base arrangement features of the genetic variation candidate site according to one embodiment of the present disclosure. As shown in FIG. 3, the foregoing step 12 includes the following steps.

At step 121, The preset site interval where the genetic variation candidate site is located is determined.

At step 122, the base arrangement features of the genetic variation candidate site are obtained according to base arrangement information of the reference genome within the preset site interval, where the base arrangement features are used for representing the base arrangement order.

In an example of embodiments of the present disclosure, each genetic variation candidate site has at least one gene sequencing read. In order to improve the accuracy of genetic variation identification, not only the base arrangement information of the genetic variation candidate site, but also base arrangement information of sites near the genetic variation candidate site can be taken into consideration. Here, the base arrangement information includes base arrangement information of a candidate genome, and in the case that the base arrangement information is the base arrangement information of the candidate genome, it is recognized that the base arrangement information of each gene sequencing read is identical, i.e., the base arrangement information of the candidate genome. Therefore, the preset site interval where in the genetic variation candidate site is located is determined according to gene position information of the genetic variation candidate site. For example, an interval formed by 150 bases before and after the genetic variation candidate site is taken as the preset site interval where the genetic variation candidate site is located. Then, the base arrangement information of the reference genome within the preset site interval is obtained for each site within the preset site interval, and the base arrangement features of the genetic variation candidate site are generated from the base arrangement information of the reference genome within the preset site interval. The base arrangement information consists of the base sequence of the reference genome at each site within the preset site interval. For example, if the preset site interval includes four base sequences, i.e., A, C, G, and T, the base arrangement information is an ACGT base arrangement order. The base arrangement features are represented by base arrangement feature vectors, and are part of a feature matrix of the genetic variation candidate site. For example, if there are four base arrangement feature vectors representing the base arrangement information, i.e., a1, a2, a3, and a4, a1, a2, a3, and a4 are the first four dimensions of features of the feature matrix.

In the example of the embodiments of the present disclosure, not only the base arrangement features corresponding to the genetic variation candidate site, but also non-base arrangement features having base arrangement invariance of the genetic variation candidate site is taken into consideration when identifying genetic variation of the genetic variation candidate site. A process of determining the non-base arrangement features of the genetic variation candidate site is explained in detail in an example below.

FIG. 4 is a flowchart of a process of determining the non-base arrangement features of the genetic variation candidate site according to one embodiment of the present disclosure. As shown in FIG. 4, the foregoing step 13 includes the following steps.

At step 131, non-base arrangement information of at least one gene sequencing read at each site within the preset site interval are obtained.

At step 132, the non-base arrangement features of the genetic variation candidate site are determined based on the non-base arrangement information at each site within the preset site interval.

In an example of embodiments of the present disclosure, considering that genetic data has a base arrangement invariance property, the non-base arrangement information of at least one gene sequencing read at each site within the preset site interval is obtained in a genetic variation identification process. Here, the non-base arrangement information is the information having the base arrangement invariance, such as the number of gene sequencing reads corresponding to a site and the number of variants. There may be multiple types of non-base arrangement information. Accordingly, the non-base arrangement feature generated from each type of non-base arrangement information may form one non-base arrangement feature vector. There may be one or more non-base arrangement feature vectors.

The genetic variation identification solution provided in the embodiments of the present disclosure may be applied to a patient diagnosed with a cancer, so that the patient is guided for medication through genetic variation identification. Therefore, some of gene sequencing reads are derived from a normal cell, and the normal cell is recognized as a cell that is not diseased. Furthermore, the other gene sequencing reads are derived from a diseased cell. Therefore, when determining non-base arrangement features of the genetic variation candidate site, the non-base arrangement sequences of the genetic variation candidate site are determined separately based on the gene sequencing reads derived from the normal cell and the gene sequencing reads derived from the diseased cell.

In one possible implementation, when determining non-base arrangement features of the genetic variation candidate site, a gene sequencing read derived from the normal cell is determined from at least one gene sequencing read, and then the non-base arrangement features of the genetic variation candidate site are determined based on the non-base arrangement information of the gene sequencing read of the normal cell at each site within the preset site interval. In this case, the non-base arrangement features of the genetic variation candidate site are determined based on the gene sequencing read derived from the normal cell.

Several examples of determining the non-base arrangement features of the genetic variation candidate site based on the gene sequencing read of the normal cell are provided below.

In an example of the embodiments of the disclosure, when determining the non-base arrangement features of the genetic variation candidate site, a first gene sequencing read having a consistent base type with the reference genome at the genetic variation candidate site is determined from the gene sequencing reads, and then the non-base arrangement features of the genetic variation candidate site are determined according to the number of first gene sequencing reads corresponding to each site within the preset site interval.

In the example, the first gene sequencing read in which no variation occurs at the genetic variation candidate site is selected from the gene sequencing reads, and for each site within the preset site interval, the number of the first gene sequencing reads at the site is counted. In other words, the number of the first gene sequencing reads containing the site is counted. The first gene sequencing read containing a site is recognized to the first gene sequencing read corresponding to the site. Because the length of each gene sequencing read may be different, the position of the gene variation candidate site with respect to each gene sequencing read is different. For example, the genetic variation candidate site may be located at a middle position of the gene sequencing read, and may also be located at an edge position of the gene sequencing position. Therefore, the number of gene sequencing reads corresponding to each site within the preset site interval is different. One non-base arrangement feature vector corresponding to the non-base arrangement feature is generated according to the number of first gene sequencing reads corresponding to each site, and each feature element in the non-base arrangement feature vector corresponds to the number of first gene sequencing reads at the corresponding site.

In another example of the embodiments of the disclosure, when determining non-base arrangement features of the genetic variation candidate site, a first gene sequencing read having a consistent base type with the reference genome at the genetic variation candidate site is determined from the gene sequencing reads; then the number of first gene sequencing reads having inconsistent base types with the reference genome is determined at each site within the preset site interval, as the number of first gene sequencing reads to which variation occurs; and the non-base arrangement features of the genetic variation candidate site are determined according to the number of first gene sequencing reads to which variation occurs.

In the example, the first gene sequencing read in which no variation occurs at the genetic variation candidate site is selected from the gene sequencing reads, and for each site within the preset site interval, the number of the first gene sequencing reads in which genetic variation occurs at the site is counted. Here, although genetic variation does not occur to the gene sequencing read at the genetic variation candidate site (i.e., the base types are consistent between the genetic variation candidate site and the reference genome), genetic variation may occur at sites other than the genetic variation candidate site (i.e., the base types are inconsistent between the other sites and the reference genome). Therefore, the number of first gene sequencing reads to which variation occurs at the site is counted. In other words, for each site, the number of first gene sequencing reads to which variation occurs at the site is counted in the first gene sequencing reads containing the site. One non-base arrangement feature vector corresponding to the non-base arrangement feature is generated according to the number of first gene sequencing reads, to which variation occurs, corresponding to each site, and each feature element in the non-base arrangement feature vector corresponds to the number of first gene sequencing reads, to which variation occurs, at the corresponding site, i.e., the number of first gene sequencing reads which contains the corresponding site and to which variation occurs at the corresponding site.

For example, for the gene sequencing reads derived from the normal cell, the first gene sequencing read to which no variation occurs at the genetic variation candidate site is determined from the gene sequencing reads of the normal cell, and then for each site within the preset site interval, the number of first gene sequencing reads corresponding to each site and the number of the first gene sequencing reads to which variation occurs at the site are counted. The two types of information correspond to a fifth dimension feature and a sixth dimension feature in the foregoing feature matrix.

In another example of the embodiments of the disclosure, when determining the non-base arrangement features of the genetic variation candidate site, a second gene sequencing read having a base type at the genetic variation candidate site consistent with a variant base type of the genetic variation candidate site is determined from the gene sequencing reads, and then the non-base arrangement features of the genetic variation candidate site are determined according to the number of second gene sequencing reads corresponding to each site within the preset site interval. In the example, the second gene sequencing reads consistent in variation with the genetic variation candidate site are selected from the gene sequencing reads, and for each site within the preset site interval, the number of the second gene sequencing reads at the site is counted. One non-base arrangement feature vector corresponding to the non-base arrangement feature is generated according to the number of second gene sequencing reads corresponding to each site, and each feature element in the non-base arrangement feature vector corresponds to the number of second gene sequencing reads at the corresponding site.

In another example of the embodiments of the disclosure, when determining non-base arrangement features of the genetic variation candidate site, a second gene sequencing read having a base type at the genetic variation candidate site consistent with a variant base type of the genetic variation candidate site is determined from the gene sequencing reads; then the number of second gene sequencing reads having inconsistent base types with the reference genome is determined at each site within the preset site interval, as the number of second gene sequencing reads to which variation occurs; and the non-base arrangement features of the genetic variation candidate site are determined according to the number of second gene sequencing reads to which variation occurs. In the example, the second gene sequencing reads consistent in variation with the genetic variation candidate site are selected from the gene sequencing reads (the variant base type of the genetic variation candidate site can be obtained through gene sequencing), and for each site within the preset site interval, the number of the second gene sequencing reads to which genetic variation occurs at the site, i.e., the number of the second gene sequencing reads which contains the site and to which vibration occurs at the site is counted. One non-base arrangement feature vector corresponding to the non-base arrangement feature is generated according to the number of second gene sequencing reads corresponding to each site, and each feature element in the non-base arrangement feature vector corresponds to the number of second gene sequencing reads at the corresponding site.

For example, for the gene sequencing reads derived from the normal cell, the second gene sequencing reads consistent in variation with the genetic variation candidate site are determined from the gene sequencing reads of the normal cell, and then for each site within the preset site interval, the number of second gene sequencing reads corresponding to each site and the number of the second gene sequencing reads to which variation occurs at the site are counted. The two types of information correspond to a sixth dimension feature and an eighth dimension feature in the foregoing feature matrix.

In another example of the embodiments of the disclosure, when determining non-base arrangement features of the genetic variation candidate site, a third gene sequencing read is determined from the gene sequencing reads, and then the non-base arrangement features of the genetic variation candidate site are determined according to the number of the third gene sequencing reads corresponding to each site within the present site interval. Here, the base type of the third gene sequencing read at the genetic variation candidate site is inconsistent with the base type of the reference genome, and the base type of the third gene sequencing read at the genetic variation candidate site is inconsistent with the variant base type of the genetic variation candidate site. That is, the third gene sequencing read refers to the gene sequencing reads other than the first gene sequencing reads and the second gene sequencing reads in the gene sequencing reads. The third gene sequencing read is the gene sequencing read in which gene insertion, gene missing, or the like exists at the genetic variation candidate site. In the example, the third gene sequencing reads left are determined from the gene sequencing reads, and for each site within the preset site interval, the number of the third gene sequencing reads at the site is counted. One non-base arrangement feature vector corresponding to the non-base arrangement feature is generated according to the number of third gene sequencing reads corresponding to each site, and each feature element in the non-base arrangement feature vector corresponds to the number of third gene sequencing reads at the corresponding site.

In another example of the embodiments of the disclosure, when determining non-base arrangement features of the genetic variation candidate site, a third gene sequencing read is determined from the gene sequencing reads; then the number of third gene sequencing reads having inconsistent base types with the reference genome is determined at each site within the preset site interval, as the number of third gene sequencing reads to which variation occurs; and the non-base arrangement features of the genetic variation candidate site are determined according to the number of third gene sequencing reads to which variation occurs. Here, the base type of the third gene sequencing read at the genetic variation candidate site is inconsistent with the base type of the reference genome, and the base type of the third gene sequencing read at the genetic variation candidate site is inconsistent with the variant base type of the genetic variation candidate site. That is, the third gene sequencing read refers to the gene sequencing reads other than the first gene sequencing reads and the second gene sequencing reads in the gene sequencing reads. In the example, the third gene sequencing reads left are determined from the gene sequencing reads, and for each site within the preset site interval, the number of the third gene sequencing reads to which genetic variation occurs at the site is counted. One non-base arrangement feature vector corresponding to the non-base arrangement feature is generated according to the number of third gene sequencing reads, to which variation occurs, corresponding to each site, and each feature element in the non-base arrangement feature vector corresponds to the number of third gene sequencing reads, to which variation occurs, at the corresponding site.

For example, for the gene sequencing reads derived from the normal cell, the third gene sequencing reads other than the first gene sequencing reads and the second gene sequencing reads are selected from the gene sequencing reads of the normal cell, and then for each site within the preset site interval, the number of third gene sequencing reads corresponding to each site and the number of the third gene sequencing reads to which variation occurs at the site are counted. The two types of information correspond to a ninth dimension feature and a tenth dimension feature in the foregoing feature matrix.

In one possible implementation, when determining non-base arrangement features of the genetic variation candidate site, a gene sequencing read derived from a diseased cell is determined from at least one gene sequencing read, and then the non-base arrangement features of the genetic variation candidate site are determined based on non-base arrangement information of the gene sequencing read of the diseased cell at each site within the preset site interval. In this case, the non-base arrangement features of the genetic variation candidate site are determined based on the gene sequencing read derived from the normal cell.

In the implementation, for the process of determining the non-base arrangement features of the genetic variation candidate site based on the gene sequencing read of the diseased cell, refer to the foregoing process of determining the non-base arrangement features based on the gene sequencing read of the diseased cell. For example, for the gene sequencing reads derived from the diseased cell, the first gene sequencing read, the second gene sequencing read, and the third gene sequencing read are determined from the gene sequencing reads of the diseased cell, and then for each site within the preset site interval, the number of the first gene sequencing reads corresponding to each site and the number of variants, the number of the second gene sequencing reads and the number of variants, and the number of the third gene sequencing reads and the number of variants are counted. The information corresponds to eleventh to sixteenth dimension features of the foregoing feature matrix.

According to the foregoing methods, non-base arrangement features of a genetic variation candidate site are determined according to non-base arrangement information of at least one gene sequencing read related to base arrangement within a preset site interval. Therefore, genetic variation identification gets easier and more accurate when base arrangement invariance of gene data is taken into consideration during genetic variation identification. A process of identifying genetic variation of the genetic variation candidate site is explained in one example below.

FIG. 5 is a flowchart of a process of identifying genetic variation of the genetic variation candidate site according to one embodiment of the present disclosure. As shown in FIG. 5, the foregoing step 14 includes the following steps.

At step 141, a feature matrix of the genetic variation candidate site is obtained according to the base arrangement features and the non-base arrangement features of the genetic variation candidate site, where first dimension features of the feature matrix correspond to the base arrangement features and the non-base arrangement features of the genetic variation candidate site, and second dimension features of the feature matrix correspond to sites within a preset site interval.

At step 142, genetic variation of the genetic variation candidate site is identified according to the feature matrix of the genetic variation candidate site.

In an example of embodiments of the present disclosure, after determining the base arrangement features and the non-base arrangement features of the genetic variation candidate site, the base arrangement features and the non-base arrangement features are subjected to feature integration by using a genetic variation identification model obtained based on a neural network, and base arrangement feature vectors formed by the base arrangement features and non-base arrangement feature vectors formed by the non-base arrangement features are combined as one feature matrix. The first dimension features of the feature matrix correspond to the base arrangement information and the non-base arrangement information, and the second dimension feature corresponds to sites within the preset site interval. The size of the feature matrix is the product of the number of feature vectors and the size of the preset site interval. For example, if there are 16 feature vectors and the preset site interval includes 150 sites, the size of the feature matrix is 16×150, where the first dimension features correspond to 16 dimensions of feature vectors, the first to fourth dimension feature vectors correspond to the base arrangement features, and the fifth to sixteenth dimension feature vectors correspond to the non-base arrangement features, which has base arrangement invariance. Then, genetic variation of the variation candidate site is identified according to the feature matrix by using the foregoing genetic variation identification model. According to the method, the base arrangement information and the non-base arrangement information corresponding to the genetic variation candidate site are integrated by using a neural network model, so that gene sequencing data is analyzed more comprehensively and genetic variation identification gets more accurate.

In one possible implementation, identifying genetic variation of the genetic variation candidate site according to an integration feature of the genetic variation candidate site includes: obtaining a variation value for occurrence of genetic variation at the genetic variation candidate site according to the feature matrix of the genetic variation candidate site, and in the case that the variation value is greater than or equal to a preset threshold, determining that generic variation exists at the genetic variation candidate site. Here, the variation value for occurrence of genetic variation indicates the possibility of true variation at the genetic variation candidate site, for example, the greater the variation value, the greater the possibility of true variation at the genetic variation candidate site. An obtained two-dimensional feature matrix is processed by using the foregoing genetic variation identification model to obtain the variation value, and whether genetic variation of the genetic variation candidate site is the true variation is determined according to the variation value. In one possible implementation, the variation value ranges from 0 to 1. The preset threshold is set, according to an application scene, as, for example, 0.3 and 0.5. if the variation value is greater than the present threshold, it is recognized that the genetic variation of the genetic variation candidate site is the true variation, i.e., genetic variation caused by a disease, otherwise, it is recognized that the genetic variation of the genetic variation candidate site is pseudo variation, i.e., a genetic abnormality formed by an interference.

In the embodiments of the present disclosure, genetic variation of a genetic variation candidate site is identified by using a genetic variation identification model. In a training process of the genetic variation identification model, matrix conversion is performed on a feature matrix extracted by the genetic variation identification model by using base arrangement invariance of genetic data. Therefore, data enhancement processing is performed in the model training process, and thus the trained genetic variation identification model has better robustness and the over-fitting problem is mitigated.

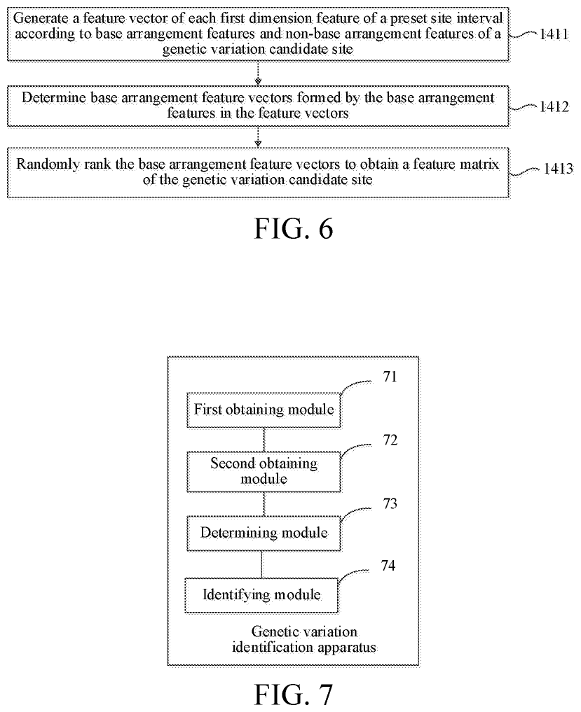

FIG. 6 is a flowchart of a process of obtaining the feature matrix of the genetic variation candidate site according to one embodiment of the present disclosure.

In embodiments of the present disclosure, data enhancement of base arrangement information can be applied to a training processing of a genetic variation identification model. As shown in FIG. 6, obtaining the feature matrix of the genetic variation candidate site according to the base arrangement features and the non-base arrangement features of the genetic variation candidate site includes the following steps.

At step 1411, a feature vector of each first dimension feature of the preset site interval is generated according to the base arrangement features and the non-base arrangement features of the genetic variation candidate site.

At step 1412, base arrangement feature vectors formed by the base arrangement features in the feature vectors are determined.

At step 1413, the base arrangement feature vectors are ranked randomly to obtain the feature matrix of the genetic variation candidate site.

Here, first dimension features correspond to base arrangement information of at least one gene sequencing read within the preset site interval, and a feature vector of the first dimension feature includes a base arrangement vector formed by the base arrangement feature and a non-base arrangement feature vector formed by the non-base arrangement feature. Because the non-base arrangement feature has base arrangement invariance, after the arrangement order of the base arrangement feature vector changes, the non-base arrangement feature will not be affected. Therefore, the base arrangement feature vectors formed from the base arrangement features in the feature vectors are ranked randomly to obtain the feature matrix of the genetic variation candidate site, so that data enhancement processing on the base arrangement information is achieved, and a trained genetic variation identification model has superior performance after the base arrangement invariance property is taken into consideration.

For example, if there are 16 feature vectors, the first dimension features correspond to 16 dimensions of feature vectors, the first to fourth dimension feature vectors correspond to the base arrangement features, and the fifth to sixteenth dimension feature vectors correspond to the non-base arrangement features, the first to fourth feature vectors are ranked randomly to form multiple feature matrices.

In the embodiments of the present disclosure, by extracting base arrangement features and non-base arrangement features of a genetic variation candidate site, base arrangement invariance of genetic data is taken into consideration during genetic variation identification, so that an identification result of genetic variation is more accurate, and the accuracy of genetic variation identification is improved by screening out germ-line genetic variation and interference caused by noise and errors.

It can be understood by persons skilled in the art that in the foregoing methods according to the specific implementations, the writing order of the steps does not imply a strict execution order to constitute any limitation on the implementation processes. The specific execution order of the steps should be determined based on functions and possible internal logic thereof.

FIG. 7 is a block diagram of a genetic variation identification apparatus according to embodiments of the present disclosure. As shown in FIG. 7, the genetic variation identification apparatus includes:

a first obtaining module 71, configured to obtain at least one gene sequencing read corresponding to a genetic variation candidate site;

a second obtaining module 72, configured to obtain base arrangement features of the genetic variation candidate site;

a determining module 73, configured to determine non-base arrangement features of the genetic variation candidate site based on non-base arrangement information of the at least one gene sequencing read within a preset site interval, where the non-base arrangement features remain unchanged after a base arrangement order changes; and

an identifying module 74, configured to identify genetic variation of the genetic variation candidate site based on the base arrangement features and the non-base arrangement features of the genetic variation candidate site.

In one possible implementation, the second obtaining module 72 includes:

a first determining sub-module, configured to determine the preset site interval where the genetic variation candidate site is located; and

a second determining sub-module, configured to obtain the base arrangement features of the genetic variation candidate site according to base arrangement information of a reference genome within the preset site interval, where the base arrangement features are used for representing the base arrangement order.

In one possible implementation, the determining module 73 includes:

a first obtaining sub-module, configured to obtain non-base arrangement information of the at least one gene sequencing read at each site within the preset site interval; and

a third determining sub-module, configured to determine the non-base arrangement features of the genetic variation candidate site based on the non-base arrangement information at each site within the preset site interval.

In one possible implementation, the third determining sub-module is specifically configured to:

determine, from the gene sequencing reads, a first gene sequencing read having a consistent base type with the reference genome at the genetic variation candidate site; and determine the non-base arrangement features of the genetic variation candidate site according to the number of first gene sequencing reads corresponding to each site within the preset site interval.

In one possible implementation, the third determining sub-module is specifically configured to:

determine, from the gene sequencing reads, a first gene sequencing read having a consistent base type with the reference genome at the genetic variation candidate site; determine the number of first gene sequencing reads having inconsistent base types with the reference genome at each site within the preset site interval, as the number of first gene sequencing reads to which variation occurs; and determine the non-base arrangement features of the genetic variation candidate site according to the number of first gene sequencing reads to which variation occurs.

In one possible implementation, the third determining sub-module is specifically configured to:

determine, from the gene sequencing reads, a second gene sequencing read having a base type at the genetic variation candidate site consistent with a variant base type of the genetic variation candidate site; and determine the non-base arrangement features of the genetic variation candidate site according to the number of second gene sequencing reads corresponding to each site within the preset site interval.

In one possible implementation, the third determining sub-module is specifically configured to:

determine, from the gene sequencing reads, a second gene sequencing read having a base type at the genetic variation candidate site consistent with a variant base type of the genetic variation candidate site; determine the number of second gene sequencing reads having inconsistent base types with the reference genome at each site within the preset site interval, as the number of second gene sequencing reads to which variation occurs; and determine the non-base arrangement features of the genetic variation candidate site according to the number of second gene sequencing reads to which variation occurs.

In one possible implementation, the third determining sub-module is specifically configured to:

determine a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; and determine the non-base arrangement features of the genetic variation candidate site according to the number of third gene sequencing reads corresponding to each site within the preset site interval.

In one possible implementation, the third determining sub-module is specifically configured to:

determine a third gene sequencing read from the gene sequencing reads, where the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; determine the number of third gene sequencing reads having inconsistent base types with the reference genome at each site within the preset site interval, as the number of third gene sequencing reads to which variation occurs; and determine the non-base arrangement features of the genetic variation candidate site according to the number of third gene sequencing reads to which variation occurs.

In one possible implementation, the third determining sub-module is specifically configured to:

determine, from the at least one gene sequencing read, a gene sequencing read derived from a normal cell; and determine the non-base arrangement features of the genetic variation candidate site based on non-base arrangement information of the gene sequencing read of the normal cell at each site within the preset site interval.

In one possible implementation, the third determining sub-module is specifically configured to:

determine, from the at least one gene sequencing read, a gene sequencing read derived from a diseased cell; and determine the non-base arrangement features of the genetic variation candidate site based on non-base arrangement information of the gene sequencing read of the diseased cell at each site within the preset site interval.

In one possible implementation, the identifying module 74 includes:

a generating sub-module, configured to obtain a feature matrix of the genetic variation candidate site according to the base arrangement features and the non-base arrangement features of the genetic variation candidate site, where first dimension features of the feature matrix correspond to the base arrangement features and the non-base arrangement features of the genetic variation candidate site, and second dimension features of the feature matrix correspond to sites within the preset site interval; and

an identifying sub-module, configured to identify genetic variation of the genetic variation candidate site according to the feature matrix of the genetic variation candidate site.

In one possible implementation, the identifying sub-module is specifically configured to:

obtain a variation value for occurrence of genetic variation at the genetic variation candidate site according to the feature matrix of the genetic variation candidate site; and

in the case that the variation value is greater than or equal to a preset threshold, determine that genetic variation exists at the genetic variation candidate site.

In one possible implementation, the generating sub-module is specifically configured to:

generate a feature vector of each first dimension feature of the preset site interval according to the base arrangement features and the non-base arrangement features of the genetic variation candidate site; determine base arrangement feature vectors formed by the base arrangement features in the feature vectors; and randomly rank the base arrangement feature vectors to obtain the feature matrix of the genetic variation candidate site.

In one possible implementation, the first obtaining module includes:

a second obtaining sub-module, configured to obtain gene sequencing reads obtained by performing gene sequencing on a somatic gene; a comparing sub-module, configured to compare base sequences of the gene sequencing reads with a base sequence of the reference genome to obtain a comparison result; a fourth determining sub-module, configured to determine the genetic variation candidate site, at which a gene abnormality exists, of the somatic gene according to the comparison result; and a third obtaining sub-module, configured to obtain at least one gene sequencing read corresponding to the genetic variation candidate site.

In some embodiments, functions provided by or modules included in the apparatus provided in embodiments of the present disclosure can be used for implementing the method described in the foregoing method embodiments. For specific implementation, refer to the description of the foregoing method embodiments. For the purpose of concision, details are not described here again.

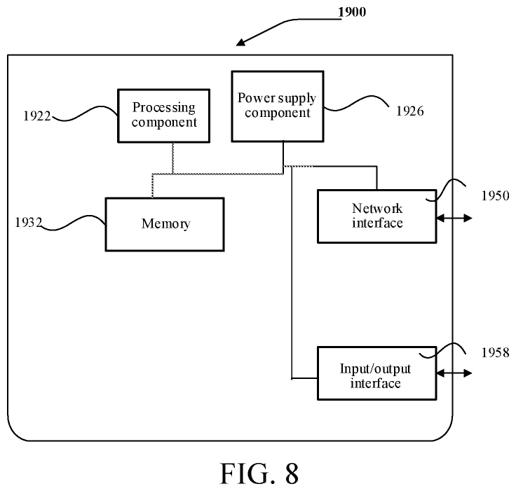

FIG. 8 is a block diagram of a genetic variation identification apparatus 1900 according to one exemplary embodiment. For example, the apparatus 1900 is provided as a server. Referring to FIG. 8, the apparatus 1900 includes a processing component 1922, and further includes one or more processors, and a memory resource as represented by a memory 1932, configured to store instructions executable for the processing component 1922, such as an application program. The application program stored in the memory 1932 includes one or more modules corresponding to a set of instructions. In addition, the processing component 1922 is configured to execute the instructions so as to execute the foregoing method.

The apparatus 1900 further includes a power supply component 1926 configured to execute power supply management of the apparatus 1900, a wired or wireless network interface 1950 configured to connect the apparatus 1900 to a network, and a Input/Output (I/O) interface 1958. The apparatus 1900 can operate an operating system stored in the memory 1932, such as Windows Server™, Mac OS X™, Unix™, Linux™, FreeBSD™ or the like.

In exemplary embodiments, a non-volatile computer-readable storage medium is further provided, for example, the memory 1932 including computer program instructions, which can be executed by the processing component 1922 of the apparatus 1900 to implement the foregoing method.

The present disclosure may be a system, a method, and/or a computer program product. The computer program product may include a computer-readable storage medium having computer-readable program instructions loaded thereon for causing a processor to carry out different aspects of the present disclosure.

The computer-readable storage medium may be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. More specific examples (a non-exhaustive list) of the computer-readable storage medium include: a portable computer diskette, a hard disk, a Random Access Memory (RAM), a Read-Only Memory (ROM), an Erasable Programmable Read-Only Memory (EPROM) or a flash memory, a Static Random Access Memory (SRAM), a portable Compact Disk Read-Only Memory (CD-ROM), a Digital Versatile Disc (DVD), a memory stick, a floppy disk, a mechanically encoded device such as punch-cards or raised structure in a groove having instructions stored thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a Local Area Network (LAN), a wide area network and/or a wireless network. The network may include copper transmission cables, optical transmission, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer program instructions for carrying out operations of the present disclosure may be assembler instructions, Instruction-Set-Architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In a scenario involving a remote computer, the remote computer may be connected to the user's computer through any type of network, including a LAN or a WAN, or the connection may be made to an external computer (for example, through the Internet using an Internet service provider). In some embodiments, electronic circuitry including, for example, programmable logic circuitry, field-programmable gate arrays (FGPAs), or programmable logic arrays (PLAs) may execute the computer-readable program instructions by utilizing state information of the computer-readable program instructions to personalize the electronic circuitry, in order to implement the different aspects of the present disclosure.

The different aspects of the present disclosure are described herein with reference to flowcharts and/or block diagrams of methods, apparatuses (systems), and computer program products according to the embodiments of the present disclosure. It should be understood that each block of the flowcharts and/or block diagrams, and combinations of the blocks in the flowcharts and/or block diagrams can be implemented by computer-readable program instructions.

These computer-readable program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can cause a computer, a programmable data processing apparatus, and/or other devices to function in a particular manner, such that the computer-readable medium having instructions stored therein includes an article of manufacture instructing instructions which implement the different aspects of the functions/acts specified in one or more blocks of the flowcharts and/or block diagrams.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus or other device implement the functions/acts specified in one or more blocks of the flowcharts and/or block diagrams.

The flowcharts and block diagrams in the accompanying drawings illustrate the architecture, functionality and operations of possible implementations of systems, methods, and computer program products according to multiple embodiments of present disclosure. In this regard, each block in the flowchart of block diagrams may represent a module, segment, or portion of instruction, which includes one or more executable instructions for implementing the specified logical function(s). In some alternative implementations, the functions noted in the block may also occur out of the order noted in the accompanying drawings. For example, two blocks shown in succession may, in fact, be executed substantially concurrently, or the blocks may sometimes be executed in the reverse order, depending upon the functionality involved. It should also be noted that each block of the block diagrams and/or flowcharts, and combinations of blocks in the block diagrams and/or flowcharts, can be implemented by special purpose hardware-based systems that perform the specified functions or acts or carried out by combinations of special purpose hardware and computer instructions.

The descriptions of the embodiments of the present disclosure have been presented for purposes of illustration, but are not intended to be exhaustive or limited to the embodiments disclosed. Many modifications and variations will be apparent to a person of ordinary skill in the art without departing from the scope and spirit of the described embodiments. The terminology used herein was chosen to best explain the principles of the embodiments, the practical application or technical improvement over technologies found in the marketplace, or to enable other persons of ordinary skill in the art to understand the embodiments disclosed herein.

1. A genetic variation identification method, comprising:
obtaining at least one gene sequencing read corresponding to a genetic variation candidate site;
obtaining base arrangement features of the genetic variation candidate site;
determining non-base arrangement features of the genetic variation candidate site based on non-base arrangement information of the at least one gene sequencing read within a preset site interval, wherein the non-base arrangement features remain unchanged after a base arrangement order changes; and
identifying genetic variation of the genetic variation candidate site based on the base arrangement features and the non-base arrangement features of the genetic variation candidate site.

2. The method according to claim 1, wherein obtaining the base arrangement features of the genetic variation candidate site comprises:
determining the preset site interval where the genetic variation candidate site is located; and
obtaining the base arrangement features of the genetic variation candidate site according to base arrangement information of a reference genome within the preset site interval, wherein the base arrangement features are used for representing the base arrangement order.

3. The method according to claim 1, wherein determining the non-base arrangement features of the genetic variation candidate site based on non-base arrangement information of the at least one gene sequencing read within the preset site interval comprises:
obtaining the non-base arrangement information of the at least one gene sequencing read at each site within the preset site interval; and
determining the non-base arrangement features of the genetic variation candidate site based on the non-base arrangement information at each site within the preset site interval.

4. The method according to claim 3, wherein determining the non-base arrangement features of the genetic variation candidate site based on the non-base arrangement information at each site within the preset site interval comprises:
determining, from the gene sequencing reads, a first gene sequencing read having a consistent base type with the reference genome at the genetic variation candidate site; and
determining the non-base arrangement features of the genetic variation candidate site according to the number of first gene sequencing reads corresponding to each site within the preset site interval.

5. The method according to claim 3, wherein determining the non-base arrangement features of the genetic variation candidate site based on the non-base arrangement information at each site within the preset site interval comprises:
determining, from the gene sequencing reads, a first gene sequencing read having a consistent base type with the reference genome at the genetic variation candidate site;
determining the number of first gene sequencing reads having inconsistent base types with the reference genome at each site within the preset site interval, as the number of first gene sequencing reads to which variation occurs; and
determining the non-base arrangement features of the genetic variation candidate site according to the number of first gene sequencing reads to which variation occurs.

6. The method according to claim 3, wherein determining the non-base arrangement features of the genetic variation candidate site based on the non-base arrangement information at each site within the preset site interval comprises:
determining, from the gene sequencing reads, a second gene sequencing read having a base type at the genetic variation candidate site consistent with a variant base type of the genetic variation candidate site; and
determining the non-base arrangement features of the genetic variation candidate site according to the number of second gene sequencing reads corresponding to each site within the preset site interval.

7. The method according to claim 3, wherein determining the non-base arrangement features of the genetic variation candidate site based on the non-base arrangement information at each site within the preset site interval comprises:
determining, from the gene sequencing reads, a second gene sequencing read having a base type at the genetic variation candidate site consistent with a variant base type of the genetic variation candidate site;
determining the number of second gene sequencing reads having inconsistent base types with the reference genome at each site within the preset site interval, as the number of second gene sequencing reads to which variation occurs; and
determining the non-base arrangement features of the genetic variation candidate site according to the number of second gene sequencing reads to which variation occurs.

8. The method according to claim 3, wherein determining the non-base arrangement features of the genetic variation candidate site based on the non-base arrangement information at each site within the preset site interval comprises:
determining a third gene sequencing read from the gene sequencing reads, wherein the base type of the third gene sequencing read at the genetic variation candidate site is inconsistent with the base type of the reference genome, and the base type of the third gene sequencing read at the genetic variation candidate site is inconsistent with the variant base type of the genetic variation candidate site; and
determining the non-base arrangement features of the genetic variation candidate site according to the number of third gene sequencing reads corresponding to each site within the preset site interval.

9. The method according to claim 3, wherein determining the non-base arrangement features of the genetic variation candidate site based on the non-base arrangement information at each site within the preset site interval comprises:
determining a third gene sequencing read from the gene sequencing reads, wherein the base type of the third gene sequencing read at the genetic variation candidate site is inconsistent with the base type of the reference genome, and the base type of the third gene sequencing read at the genetic variation candidate site is inconsistent with the variant base type of the genetic variation candidate site;
determining the number of third gene sequencing reads having inconsistent base types with the reference genome at each site within the preset site interval, as the number of third gene sequencing reads to which variation occurs; and
determining the non-base arrangement features of the genetic variation candidate site according to the number of third gene sequencing reads to which variation occurs.

10. The method according to claim 3, wherein determining the non-base arrangement features of the genetic variation candidate site based on the non-base arrangement information at each site within the preset site interval comprises:
determining, from the at least one gene sequencing read, a gene sequencing read derived from a normal cell; and
determining the non-base arrangement features of the genetic variation candidate site based on non-base arrangement information of the gene sequencing read of the normal cell at each site within the preset site interval.

11. The method according to claim 3, wherein determining the non-base arrangement features of the genetic variation candidate site based on the non-base arrangement information at each site within the preset site interval comprises:
determining, from the at least one gene sequencing read, a gene sequencing read derived from a diseased cell; and
determining the non-base arrangement features of the genetic variation candidate site based on non-base arrangement information of the gene sequencing read of the diseased cell at each site within the preset site interval.

12. The method according to claim 1, wherein identifying the genetic variation of the genetic variation candidate site based on the base arrangement features and the non-base arrangement features of the genetic variation candidate site comprises:
obtaining a feature matrix of the genetic variation candidate site according to the base arrangement features and the non-base arrangement features of the genetic variation candidate site, wherein first dimension features of the feature matrix correspond to the base arrangement features and the non-base arrangement features of the genetic variation candidate site, and second dimension features of the feature matrix correspond to sites within the preset site interval; and
identifying the genetic variation of the genetic variation candidate site according to the feature matrix of the genetic variation candidate site.

13. The method according to claim 12, wherein identifying the genetic variation of the genetic variation candidate site according to the feature matrix of the genetic variation candidate site comprises:
obtaining a variation value for occurrence of genetic variation at the genetic variation candidate site according to the feature matrix of the genetic variation candidate site; and
in the case that the variation value is greater than or equal to a preset threshold, determining that genetic variation exists at the genetic variation candidate site.

14. The method according to claim 12, wherein obtaining the feature matrix of the genetic variation candidate site according to the base arrangement features and the non-base arrangement features of the genetic variation candidate site comprises:
generating a feature vector of each first dimension feature of the preset site interval according to the base arrangement features and the non-base arrangement features of the genetic variation candidate site;
determining base arrangement feature vectors formed by the base arrangement features in the feature vectors; and
randomly ranking the base arrangement feature vectors to obtain the feature matrix of the genetic variation candidate site.

15. The method according to claim 1, wherein obtaining at least one gene sequencing read corresponding to the genetic variation candidate site comprises:
obtaining gene sequencing reads obtained by performing gene sequencing on a somatic gene;
comparing base sequences of the gene sequencing reads with a base sequence of the reference genome to obtain a comparison result;
determining the genetic variation candidate site, at which a gene abnormality exists, of the somatic gene according to the comparison result; and
obtaining at least one gene sequencing read corresponding to the genetic variation candidate site.

16. A genetic variation identification apparatus, comprising:
a processor; and
a memory configured to store processor-executable instructions,
wherein the processor is configured to invoke the instructions stored in the memory, so as to:
obtain at least one gene sequencing read corresponding to a genetic variation candidate site;
obtain base arrangement features of the genetic variation candidate site;
determine non-base arrangement features of the genetic variation candidate site based on non-base arrangement information of the at least one gene sequencing read within a preset site interval, wherein the non-base arrangement features remain unchanged after a base arrangement order changes; and
identify genetic variation of the genetic variation candidate site based on the base arrangement features and the non-base arrangement features of the genetic variation candidate site.

17. The apparatus according to claim 16, wherein obtaining base arrangement features of the genetic variation candidate site comprises:
determining the preset site interval where the genetic variation candidate site is located; and
obtaining the base arrangement features of the genetic variation candidate site according to base arrangement information of a reference genome within the preset site interval, wherein the base arrangement features are used for representing the base arrangement order.

18. The apparatus according to claim 16, wherein determining the non-base arrangement features of the genetic variation candidate site based on non-base arrangement information of the at least one gene sequencing read within the preset site interval comprises:
obtaining non-base arrangement information of the at least one gene sequencing read at each site within the preset site interval; and
determining the non-base arrangement features of the genetic variation candidate site based on the non-base arrangement information at each site within the preset site interval.

19. The apparatus according to claim 18, wherein determining the non-base arrangement features of the genetic variation candidate site based on the non-base arrangement information at each site within the preset site interval comprises:
determining, from the gene sequencing reads, a first gene sequencing read having a consistent base type with the reference genome at the genetic variation candidate site; and
determining the non-base arrangement features of the genetic variation candidate site according to the number of first gene sequencing reads corresponding to each site within the preset site interval.

20. A non-transitory computer-readable storage medium, having computer program instructions stored thereon, wherein when the computer program instructions are executed by a processor, the processor is caused to perform the operations of:
obtaining at least one gene sequencing read corresponding to a genetic variation candidate site;
obtaining base arrangement features of the genetic variation candidate site;
determining non-base arrangement features of the genetic variation candidate site based on non-base arrangement information of the at least one gene sequencing read within a preset site interval, wherein the non-base arrangement features remain unchanged after a base arrangement order changes; and
identifying genetic variation of the genetic variation candidate site based on the base arrangement features and the non-base arrangement features of the genetic variation candidate site.
